(19) United States
(12) Patent Application Publication
(10) Pub. No.: US 2025/0360289 A1
(43) Pub. Date: Nov. 27, 2025

(54) ROLLING MEMBRANE CATHETER WITH INFLATING TUBE

(71) Applicant: BIOTRONIK AG, Buelach (CH)

(72) Inventor: Morten Klabunde, Winterthur (CH)

(21) Appl. No.: 19/108,352

(22) PCT Filed: Sep. 1, 2023
(86) PCT No.: PCT/EP2023/074070

§ 371 Date: Mar. 3, 2025

(30) Foreign Application Priority Data
Sep. 7, 2022 (EP) 22194345.9

(51) Int. Cl. A61M 25/01; A61M 25/00

(52) U.S. Cl. CPC A61M 25/0119; A61M 2025/0059

(57) ABSTRACT

A catheter includes an outer shaft, an inner shaft, a rolling membrane and an inflatable element that defines an inflatable volume. The inner shaft includes an inner shaft lumen and the inner shaft is at least partially arranged within the outer shaft. The rolling membrane is connected to the outer shaft. The inflatable element includes at least two proximal ends connected to the inner shaft and at least one distal end connected to the rolling membrane.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2023/074070, which was filed Sep. 1, 2023, which application claimed priority from EP Application 22194345.9, which was filed Sep. 7, 2022.

### FIELD OF THE INVENTION

A field of the invention concerns catheters.

### BACKGROUND

Life circumstances (e.g., changed nutrition habits, increased exposition to stressful situations, etc.) of many people in modern societies have led to an increase of coronary diseases which is accompanied by an increasing number of catheter-based interventions to either contribute to an ideally early diagnosis of such diseases and/or to curatively address said diseases (e.g., by a stent implantation).

One type of catheters are catheters with rolling membranes. A rolling membrane can be understood as a hose-like flexible and tubular element wherein one end of the element (e.g., the distal end as seen in a rolled-out state) is at least partially folded into itself. Thus, an inner lumen can be formed which can extend from a proximal side of the rolling membrane catheter to a distal end of the rolling membrane catheter, for example. As a result of filling a fluid into the rolling membrane catheter from a proximal side of the catheter, for example, the rolling membrane can at least partially be unfolded, i.e., rolled out in an, e.g., distal direction, along an axis of the catheter. Due to its flexible properties, the rolling membrane can be able to roll out along an axis of a blood vessel and, due to its rolling motion, can reach even branched and/or thin arteries. The rolling membrane can, due to its unfolding, propagate along the walls of the blood vessels in a rolling manner (e.g. such that friction with the vessel walls is minimized).

However, these catheters often have the disadvantage that a distal portion of the catheter can not under all circumstances remain in a stable shape and particularly an inner shaft to which the rolling membrane can be connected can tend to kink (e.g., in curved and/or narrow and/or partially occluded artery portions). Kinking can then disadvantageously affect the properties of the catheter such that, e.g., a potential inner lumen (which can, e.g., be used for the delivery of medical drugs and/or medical devices) can be blocked such that the respective drug and/or medical device can not be delivered to an area of interest anymore.

The risk of kinking of a catheter can at least partially be avoided by providing the respective catheter with a stable inner tube or shaft thus providing the catheter with additional kinking resistance. Additional kinking resistance can, e.g., be provided by using a coil which can be wound about the respective portions of the catheter. Additionally or alternatively, it can also be possible to use braids, filaments etc. inside the inner lumen. Such a catheter can, however, under some circumstances then become too stiff for certain applications. Moreover, providing the catheter with additional kinking-resistance can also disadvantageously affect the manufacturing costs for a respective catheter.

Additionally or alternatively, it can be possible to provide a catheter with improved kinking-resistance by increasing a wall thickness of the catheter. However, as a result of the increased wall thickness of the catheter, the catheter can also not be able to access narrow blood vessels of a patient and can thus be unsuitable for certain applications (e.g., if an investigation of narrow blood vessels is required).

### SUMMARY OF THE INVENTION

A catheter of a preferred embodiment includes an outer shaft, an inner shaft, a rolling membrane and an inflatable element that defines an inflatable volume. The inner shaft includes an inner shaft lumen and the inner shaft is at least partially arranged within the outer shaft. The rolling membrane is connected to the outer shaft. The inflatable element includes at least two proximal ends connected to the inner shaft and at least one distal end connected to the rolling membrane.

### BRIEF DESCRIPTION OF THE DRAWINGS

In the following embodiments of the present invention as well as further features and embodiments of the present invention shall be described with reference to the Figures, wherein:

FIG. 1 Illustration of an exemplary catheter with an inflatable element.

FIG. 2 Illustration of an exemplary catheter with an inflatable element having welding points

### DETAILED DESCRIPTION OF THE PREFERRED EMBODIMENTS

A preferred embodiment catheter includes an inner shaft, a rolling membrane and an inflatable element and optionally an outer shaft, wherein the inflatable element includes at least two proximal ends connected to the inner shaft. The inflatable element can include an inflatable volume. The inner shaft can include an inner shaft lumen.

In a first embodiment a catheter is described including an outer shaft, an inner shaft, a rolling membrane and an inflatable element including an inflatable volume; wherein the inner shaft includes an inner shaft lumen and the inner shaft is at least partially arranged within the outer shaft and the rolling membrane is connected to the outer shaft; and wherein the inflatable element includes at least two proximal ends connected to the inner shaft and at least one distal end connected to the rolling membrane.

In a second embodiment a catheter is described including an inner shaft, a rolling membrane, an inflatable element including an inflatable volume, and no outer shaft, wherein the inner shaft includes an inner shaft lumen and the inner shaft is surrounded by the rolling membrane, and wherein the inflatable element includes at least two proximal ends connected to the inner shaft and at least one distal end connected to the rolling membrane.

This can allow a catheter with an additional and adjustable stabilizing element, namely the inflatable element. The inflatable element can for example be adapted to be in a non-inflated state when the catheter is inserted and/or pushed along a blood vessel of a patient such that maximum flexibility is provided. It can then later be transformed into an inflated state once the device is positioned at the desired location within the patient (e.g. within a blood vessel of the patient). An inner shaft lumen can be used for the delivery of additional interventional devices. When inflated, the inflatable element can provide the catheter with additional kink-resistance as the inflatable volume (e.g. fluid filled) of the inflatable element can counteract any deformation forces acting onto the inflatable element. As a result, the risk for a blocking the inner shaft lumen, e.g., due to a kinking of the catheter can be suppressed and a well-defined diameter of the inner shaft lumen can be ensured in a cost-efficient manner as no coils, etc. are required for stabilization of, e.g., the inner shaft lumen. Also, the catheter can provide the required stability without excessive wall thickness, such that it can for example be provided as a four French product.

An (operative) connection can be provided for example as a direct connection. However, an operative connection can imply that the inflatable element can move when the inner shaft or the outer shaft is moved.

In some embodiments it can be sufficient if the inflatable element is generally connected to the inner shaft and to the outer shaft without necessarily requiring at least two proximal ends connected to the inner shaft and at least one distal end connected to the outer shaft.

In some embodiments, the inner shaft can be concentrically arranged within the outer shaft.

An at least partial arrangement of the inner shaft within the outer shaft can be understood as arranging the inner shaft within the outer shaft such that at least a proximal end portion of the inner shaft is located within the outer shaft. In some cases, at least a distal (end) portion of the inner shaft can be adapted to lie outside the outer shaft (in a distal direction).

The rolling membrane and/or the inflatable element can be made from a biocompatible material, preferably a thermoplastic material, e.g. an (organic) thermoplastic polymer or thermoplastic elastomer (which can, e.g., also be used for balloons for applications in angioplasty). The rolling membrane and/or the inflatable element can be made from a pressure resistant and/or bendable and/or ductile material which can be a material (or material composition) exemplarily selected from the group of polyamides, polyurethanes, poly (dodecano-12-lactam), polyether block amides or a thermoplastic polyurethanes or mixtures thereof.

The outer shaft and/or the inner shaft can be made of a biocompatible material, preferably a polymer, a reinforced polymer or alternatively a metal or metal alloy. The material of the outer shaft and/or the inner shaft can have a higher stiffness than the material of the rolling membrane and/or the inflatable element. Preferably, the materials of which the outer shaft and the inner shaft are made can each have a higher stiffness than the materials of which the rolling membrane and the inflatable element are made. The material of which the outer shaft and the material of which the inner shaft is made each can have a higher wall thickness than the materials of which the rolling membrane and the inflatable element are made. The inflatable element, at least, when inflated, can extend along a longitudinal axis of the catheter by 20 to 40 cm, preferably by 26 to 35 cm.

In some examples, the inflatable element can be adapted such that its extension along a longitudinal axis exceeds a radial extension of the inflating element upon an inflation.

The extension of the inflatable element when inflated can allow a compact catheter design in an initial non-inflated state (e.g., with respect to a radius of the catheter) of the inflatable element such that a simple movement (e.g., with minimized friction) of the catheter to an area of interest in a blood vessel of the patient can be facilitated. The forward movement (e.g., in a distal direction) of the catheter along an axis of a blood vessel of the patient can at the same time not be limited by a large outer radius of the catheter. In other words, at the beginning of a catheter-based treatment, the inflatable element and/or the inner shaft and/or the outer shaft can be crimped to a smallest possible diameter (e.g., by evacuating an inner volume of the inflatable element) and can only be inflated if the desired area of interest is reached. Moreover, the extension of the inflatable element by 20 to 40 cm can further allow a stabilization of the catheter in a comparably large portion of the catheter such that, e.g., at least 50%, at least 70% or substantially 100% of the length of the catheter can be stabilized.

More specifically, the extension along a longitudinal axis can be at least two times, three times, four times, five times, six times, seven times, eight times, nine times or ten times the extension of the catheter along the radial direction (in an inflated state). It is further emphasized that the longitudinal extension can exceed the radial extension by more than ten times.

The inflatable element can be configured such as to provide a wall thickness, when inflated, of 0.02 to 2 mm, or 0.05 to 0.5 mm, preferably of 0.1 to 0.2 mm. The wall thickness can be substantially constant.

The inflatable element can be adapted such that the inflatable element has a thicker wall thickness in an inflated state as compared to the non-inflated state (e.g., the thickness in the inflated state can be two times, three times, four times, five times, six times, etc. larger than in the non-inflated state). Alternatively, it can also be possible that the wall thickness in the aforementioned range is provided both in the inflated and the non-inflated state.

By adapting the inflatable element such that it acquires a wall thickness in the above-mentioned range, it can be facilitated that the inflatable element only contributes to the total outer diameter of the catheter to at most a negligible extent, facilitating a small-dimension catheter with a large inner shaft lumen.

The inflatable element can be adapted such that it can at least partially be rolled-out in a distal direction of the catheter.

In some applications, the inflatable element can be adapted such that it can be folded inwards into itself, similar to a rolling membrane, such that the inflatable element can be rolled out, e.g. as a result of or supported by an inflating of the inflatable element.

The inflatable element can be adapted, e.g. as a rolling membrane, such that it preferably rolls out along a longitudinal axis of the catheter, preferably in a distal direction.

By adapting the inflatable element such that it can be rolled out, the volume the inflatable element occupies in a non-inflated state can be minimized. Therefore, it can be ensured that the inflatable element can not limit the applications of the catheter in such a way that the catheter cannot be pushed into certain areas of interest which can have a rather narrow inner diameter.

The inflatable element can include an outer cylindrical element and an inner cylindrical element wherein the inner cylindrical element can at least in part be arranged within the outer cylindrical element such that the inflatable volume is defined as a hollow cylinder volume therebetween.

At least one of the outer cylindrical element and/or the inner cylindrical element can be made from a sheet-like element (e.g., a membrane) formed into a cylinder shape.

The inner and outer cylindrical elements can in some examples also be integrally formed from a single sheet-like element. In other cases, at least one of the outer cylindrical element and/or the inner cylindrical element can be provided as a molded and/or integral cylindrical element (e.g., made from a moldable plastic material).

The inner cylindrical element can be concentrically arranged in the outer cylindrical element. The hollow cylinder can be circumferent to an inner shaft lumen of the catheter, only being separated from the inner shaft lumen by the inner cylindrical element.

By providing the outer cylindrical element and the inner cylindrical element such that a hollow cylinder is formed therebetween, a cylinder shaped inflation volume of the inflatable element can be defined. Therefore, a symmetric extension of the inflatable element along the longitudinal axis of the catheter and also along a radial direction of the inflatable element can be facilitated.

The proximal ends of the inner and outer cylindrical elements can be designed in an open manner, e.g. they can not be connected to each other. The proximal ends of the inner and outer cylindrical elements can thus form two proximal ends of the inflatable element. For example, the proximal ends of the inner and outer cylindrical elements can be connected to the inner shaft.

Each of the inner cylindrical element and the outer cylindrical element can include a distal end, and wherein the distal ends can be connected to each other. The connecting of the inner cylindrical element to the outer cylindrical element can be liquid-tight, preferably fluid-tight. A liquid-tight or fluid-tight connecting can be understood as a connecting which can prevent an escape of liquid and fluid, respectively, through the connecting, such that the inflatable volume can be inflated by the fluid/liquid. The connected distal ends of the cylindrical elements can be connected to the outer element, as described herein.

Instead of being connected at their distal ends, the inner and outer cylindrical element can also be integrally formed at the distal end. For example, a sheet like element can be folded onto itself. Subsequently, the folded element can be arranged cylindrically, wherein the cylinder axis runs perpendicular to the direction of the seam or notch of the folded sheet like element. Hence, the seam or notch can form an integral distal end of the inflatable element, and at the opposing side, two proximal ends can be formed.

The inner cylindrical element and the outer cylindrical element can be connected to each other proximal to the at least one distal end of the inflatable element.

Proximal to a distal end of the inflatable element can be understood as a location separated from the distal end of the inflatable element by at least 1 cm, 2 cm, 3 cm, 5 cm, 10 cm, or at least 20 cm in a proximal direction. By connecting the inner cylindrical element to the outer cylindrical element, a substantially constant wall thickness of the hollow cylinder (as measured along a radial direction from the outer side of the inner cylindrical element and an inner side of the outer cylindrical element) formed in between the inner cylindrical element and the outer cylindrical element can be ensured. Therefore, an asymmetric inflation of the inflatable element can be suppressed.

The inflatable element includes at least two, preferably 3, 5 or more, fixation points and/or at least one fixation seam arranged at least in part along a longitudinal axis (L) of the catheter. The at least two fixation points can be separated from each other by 2 to 5 mm. For example, the inflatable element can include 5 fixation points (e.g. welding points) or more radially spaced 72 degree apart from each other at a (longitudinal) distance of 3 mm to 5 mm. For example, two fixation seams are arranged at least in part along a longitudinal axis (L) of the catheter.

The inner cylindrical element can be connected to the outer cylindrical element at at least two fixation points, wherein the at least two fixation points can be separated from each other by 2 to 5 mm.

By providing the inflatable element or the inner cylindrical element and the outer cylindrical element with at least two fixation points, an asymmetric inflation of the inflatable element can be avoided as the radial distance between the outer side of the inner cylindrical element and the inner side of the outer cylindrical element can be kept constant. This can avoid that the inner shaft lumen of the catheter can be blocked as a result of (asymmetrically) inflating the inflatable element. Between fixation points, there can not be any connection between inner and outer cylindrical elements, such that the fluid can freely propagate between the inner and outer cylindrical elements.

In some applications, the inflatable element can be provided with more than two fixation points, e.g., with four, eight, twelve, etc. fixation points. The multiple fixation points can be arranged in a pattern.

The inner cylindrical element can be connected to the outer cylindrical element by at least one fixation seam arranged at least in part along a longitudinal axis of the catheter, preferably by at least two fixation seams. This can ensure particular control of the wall thickness, and the inflation process can be particularly well controlled as well. It can particularly be ensured that the separation of the inner circular element and the outer circular element is kept constant with respect to each other.

If at least two fixation seams are implemented, such as, e.g., three fixation seams, the fixation seams can be separated from each other by a same angular separation as seen from a cut through the inflatable element perpendicular to the longitudinal axis of the catheter. The seams can run parallel to the longitudinal axis of the catheter. However, it can also be considered that they include a spiral shape, e.g. spiraling along the inner and outer cylindrical elements.

The connecting can be provided by welding. The welding can be based on applying radiation (e.g., a laser radiation) onto two respective overlapping portions (preferably touching each other) of the inner cylindrical element and the outer cylindrical element. The radiation source can be adapted such that its emitted radiation can be sufficient to at least locally heat the overlapping portions to such an extent that they locally melt and form a connection with each other. Additionally or alternatively, the welding can also be based on friction welding and/or hot air welding. In some cases, the connecting can additionally or alternatively be established by gluing.

By providing the connecting by welding, a durable and cost-efficient connecting can be provided.

The inner shaft can include a first tubular element and a second tubular element, wherein the first tubular element can be arranged within the second tubular element thereby defining an annular lumen. The first tubular element can be arranged concentrically within the second tubular element. The first tubular element and the second tubular element can be provided with hollow inner lumens. In some cases, the first tubular element can be movable relative to the second tubular element along the longitudinal axis of the catheter.

At least one of the first tubular element and the second tubular element can be made from a flexible material. In other cases, at least one of the first tubular element and the second tubular element can be made from a rigid, non-flexible material (e.g., a metal or any other suitable material). The annular lumen can preferably be in the shape of a hollow cylinder arranged in between the first tubular element and the second tubular element.

The inflatable volume of the inflatable element can be in fluid communication with the annular lumen.

In an example, the first tubular element can be connected to the inner cylindrical element of the inflatable element, e.g. to a proximal end of the inner cylindrical element. In some cases, the second tubular element can be connected to the outer cylindrical element of the inflatable element, e.g. to a proximal end of the outer cylindrical element. Hence, two proximal ends of the inflatable element can be connected to the inner shaft. The connection of the inflatable element to both the first tubular element and the second tubular element can thus allow a simplified inflation and deflation procedure of the inflatable element from a proximal end of the catheter and from a location outside of the body of the patient.

The inflatable element can be connected to the distal end of the inner shaft. The inner cylindrical element can be connected to the first tubular element and the outer cylindrical element can be connected to a second tubular element. Preferably, a proximal end of the inner cylindrical element can be connected to a distal end of the first tubular element and a proximal end of the outer cylindrical element can connected to a distal end of the second tubular element.

By the aspects described herein, an inflatable element can be provided that provides an inner inflatable lumen with a predetermined inner diameter in a dimensionally stable manner.

The catheter further includes a rolling membrane, wherein the at least one distal end of the inflatable element can be connected to the rolling membrane.

A rolling membrane can be understood as a hose-like flexible and tubular element wherein one end of the element (e.g., the distal end as seen in a rolled-out state) is at least partially folded into itself. Thus, a (hollow) inner rolling membrane lumen can be formed which can extend from a proximal side of the rolling membrane catheter to a distal end of the rolling membrane catheter, for example. As a result of filling a fluid into the rolling membrane catheter from a proximal side of the catheter, for example, the rolling membrane can at least partially be unfolded, i.e., rolled out in an, e.g., distal direction, along an axis of the catheter. Due to its flexible properties, the rolling membrane can be able to roll out along an axis of a blood vessel and, due to its rolling motion, can reach even branched and/or thin arteries. The rolling membrane can, due to its unfolding, propagate along the walls of the blood vessels in a rolling manner (e.g. such that friction with the vessel walls is minimized).

The rolling membrane can be provided as a hose-like element with an inner rolling membrane lumen wherein at least a portion, preferably a distal portion (as seen when the rolling membrane is rolled out) of the hose-like element is folded inwards into itself. The rolling membrane can preferably be adapted such that it can be rolled out along a longitudinal direction (along a distal direction) of the catheter.

The at least one distal end of the inflatable element can be connected to the distal end (as seen when the rolling membrane is rolled out) of the rolling membrane (the distal end of the rolling membrane can also be understood as the most proximal inner portion of the rolling membrane as it is rolled out).

By providing the catheter with a rolling membrane, a simplified movement of the distal end of the catheter to an area of interested through a blood vessel of the patient can be supported. This decreases the control and stirring complexity of the catheter along the blood vessel of the patient and can thus also contribute to a reduction of the treatment complexity. If the area of interested is reached by the distal end of the rolling membrane, the rolling membrane can experience further stabilization by the inflatable element. such that a kinking of the rolling membrane can be avoided. Due to the inflation the inner lumen grows to the desired size and allows enough space for further devices. Kinking but also deformation which leads to the flattening of the inner lumen are avoided.

The rolling membrane can be connected to the outer shaft. For example, a proximal end of the rolling membrane (when the rolling membrane is rolled out) can be connected to the outer shaft.

The rolling membrane can be rolled out by providing a liquid or a fluid (e.g., from a proximal direction of the catheter) to an annular lumen defined in between an inner side of the outer shaft and an outer side of the second tubular element of the inner shaft. Additionally or alternatively, it can be rolled-out by moving the inner shaft distally and/or moving the outer shaft (or, if no separate outer shaft is provided, the proximal end of the rolling membrane) proximally.

The inflatable volume of the inflatable element can be adapted to allow a delivery of a medical drug and/or a medical device from a proximal portion of the catheter to a distal portion of the catheter.

By providing the inner shaft lumen such that it can allow the delivery of a medical drug and/or a medical device (e.g. stent), the versatility of the catheter can be increased. Due to the inflatable element (set to a non-inflated state in the beginning of a catheter-based intervention), the catheter can be pushed into a narrow blood vessel of the patient (e.g., into a blood vessel with an inner diameter similar to the outer diameter of the catheter). Therefore, even narrow blood vessels can be reached by the catheter. Prior to a potential stent implantation/drug delivery, the catheter can further be stabilized by inflating the inflatable element. At the same time, a secure inner channel is provided by the inner lumen of the inflatable element which can provide a predetermined diameter (e.g. for delivering four French devices). The predetermined inner diameter can be in the range of 0.5 mm to 2 mm, 0.8 mm to 1.8 mm, 1 mm to 1.5 mm.

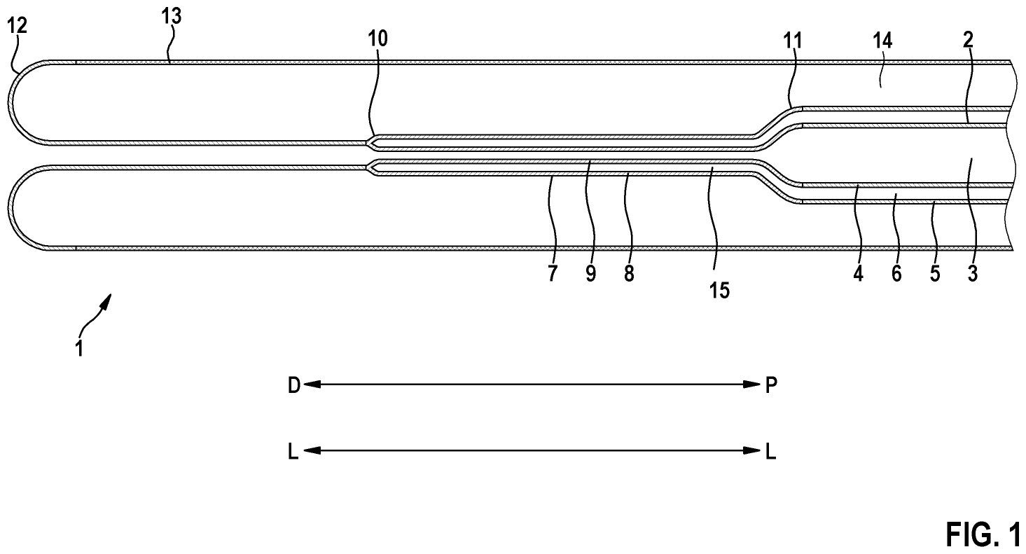

FIG. 1 exemplarily shows a catheter 1 including an outer shaft 13, an inner shaft 2, a rolling membrane 12 and an inflatable element 7. The inner shaft 2 is at least partially arranged within the outer shaft 13. The inflatable element 7 includes at least two proximal ends 11 connected to the inner shaft 2 and at least one distal end 10 connected to the rolling membrane 12.

The inner shaft 2 can be located at a proximal end P of the catheter 1. The inner shaft 2 includes an inner shaft lumen 3. The inner shaft lumen 3 extends from a proximal end of inner shaft 2 to a distal end of inner shaft 2.

The inner shaft 2 can include a first tubular element 4 and a second tubular element 5 wherein the first tubular element 4 can concentrically be arranged within the second tubular element 5. Between the first tubular element 4 and the second tubular element 5, an annular lumen 6 can be defined. First and second tubular elements 4, 5 can be formed by inner shafts having different diameters.

The inflatable element 7 is preferably arranged at the distal end of the inner shaft 2. The inflatable element 7 includes an inflatable volume 15.

The inflatable element 7 can include an outer cylindrical element 8 and an inner cylindrical element 9, wherein the outer cylindrical element 8 can at least connected to the inner cylindrical element 9 at a distal end 10 of the inflatable element 7.

Preferably, the proximal end 11 of the inflatable element 7 is connected to the distal end of the inner shaft 2. The connection can be provided such that a fluid communication of the inflatable volume of inflatable element 7 with the annular lumen 6 is established. Moreover, an inner inflatable lumen of inflatable element 7 can be in fluid communication with the inner shaft lumen 3 of inner shaft 2.

In FIG. 1, the proximal ends of each of the outer cylindrical element 8 and the inner cylindrical element 9 can be connected to the inner shaft. Specifically, the proximal end of the outer cylindrical element 8 can be connected to the second tubular element 5 of inner shaft 2. The proximal end of the inner cylindrical element 9 can be connected to the first tubular element 4 of inner shaft 2.

In FIG. 1, the inflatable element 7 is only partially inflated. When further inflated, the inflatable element 7 can predominantly extend along a longitudinal axis L of the catheter, preferably in the distal direction D.

The catheter 1 can further include a rolling membrane 12. The rolling membrane 12 is connected to the inflatable element 7, e.g. to the distal end 10 of inflatable element 7.

When the rolling membrane 12 is rolled out, the point of connection between distal end 10 of inflatable element 7 and rolling membrane 12 can form a distal end of the catheter.

At its opposing end, the rolling membrane 12 can be connected to a distal end of an outer shaft 13.

An inner lumen 14 is formed in between the outer shaft 13 and the inner shaft 2. By providing a fluid to the inner lumen 14, preferably from a proximal end P of the catheter 1, a roll out of the rolling membrane 12 can be initiated. The rolling membrane 12 can be adapted to roll out along the distal direction D and the longitudinal axis L of the catheter 1. The rolling out can be accompanied by a distal movement of inner shaft 2 and/or a proximal movement of outer shaft 13.

For example, once the rolling membrane 13 has been rolled out, inflatable element 7 can be inflated. This can ensure stability of the catheter 1. It also can ensure that an inner lumen (including that of the inflatable element 7) is provided to safely deliver a drug and/or device to the distal end of the catheter 1. For example, inflatable element 7 can be adapted such that, in an inflated state, its inner lumen can include a diameter that is substantially identical to an inner diameter of the inner shaft lumen 3 of inner shaft 2.

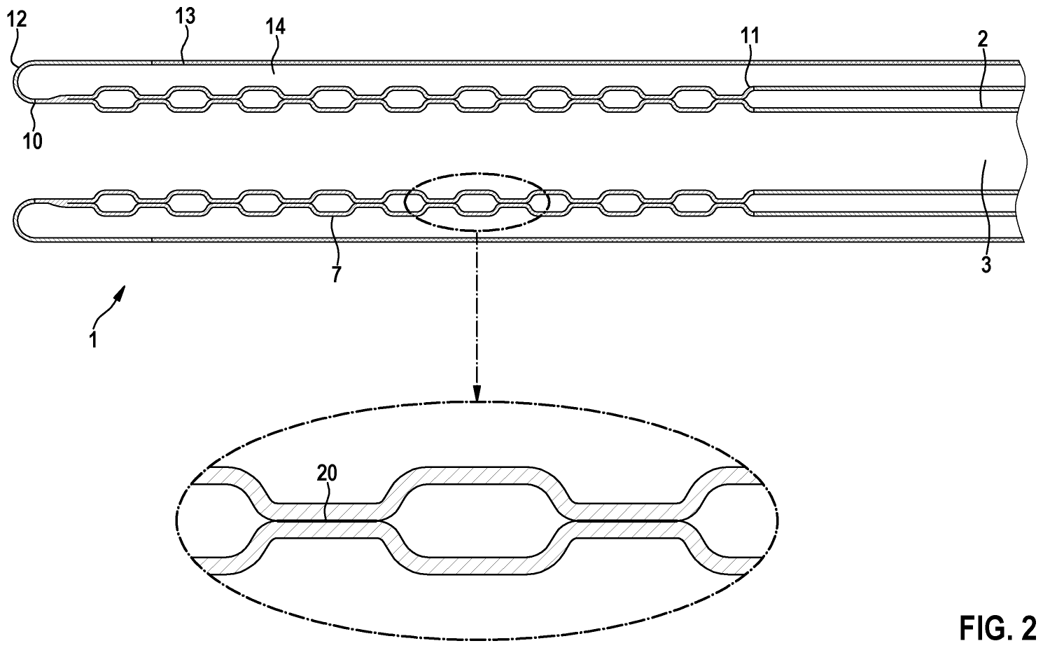

FIG. 2 shows a catheter 1 including an outer shaft 13, an inner shaft 2, a rolling membrane 12 and an inflatable element 7.

The inner shaft 2 is at least partially arranged within the outer shaft 13. The inner shaft 2 that can be located at a proximal end of the catheter 1. The inner shaft 2 includes an inner shaft lumen 3. The inner shaft lumen 3 extends from a proximal end of inner shaft 2 to a distal end of inner shaft 2.

At a distal end of the inner shaft 2, the inflatable element 7 is arranged. The inflatable element 7 includes an inflatable volume. The inflatable element 7 includes at least two proximal ends 11 and at least one distal end 10. The rolling membrane 12 is at least connected to a distal end 10 of the inflatable element 7. The proximal end 11 of the inflatable element 7 is connected to the distal end of the inner shaft 2. The inflatable volume of inflatable element 7 is in fluid communication with the inner shaft lumen 3 of inner shaft 2.

The rolling membrane 12 is connected to the inflatable element 7, e.g. to the distal end 10 of inflatable element 7. When the rolling membrane 12 is rolled out, the point of connection between distal end 10 of inflatable element 7 and rolling membrane 12 can form a distal end of the catheter. At its opposing end, the rolling membrane 12 is connected to a distal end of an outer shaft 13.

An inner lumen 14 is formed in between the outer shaft 13 and the inner shaft 2. By providing a fluid to the inner lumen 14, preferably from a proximal end of the catheter 1, a roll out of the rolling membrane 12 can be initiated. The rolling membrane 12 can be adapted to roll out along the distal direction D and the longitudinal axis L of the catheter 1. The rolling out can be accompanied by a distal movement of inner shaft 2 and/or a proximal movement of outer shaft 13.

The inflatable element includes at least two fixation points 20 and/or at least one fixation seam arranged at least in part along a longitudinal axis (L) of the catheter.

Further exemplary embodiments are provided below.

A method can be provided for operating a catheter having an inflatable element with an inflatable volume. The catheter can be any catheter as described herein. The method can include the following steps: Inserting the catheter into a cavity, such as a vessel, of a patient. Inflating the inflatable volume of the inflatable element, preferably after having inserted the catheter.

In some examples, the further step of delivering a medical device and/or a substance through the inner lumen of the inflatable element can be provided, preferably after having inflated the inflatable volume of the inflatable element.

In some examples, the step of inserting can include inserting the catheter into the vessel with the inflatable element in a deflated state. In some examples, the step of inflating the inflatable volume can include inflating after the catheter has reached a pre-determined (final) position in the cavity, e.g., vessel.

In some examples, the method can further include the step of manipulating the catheter such that the inner lumen of the inflatable element extends along a longitudinal axis of the catheter and to a distal end of the catheter. For example, a rolling membrane connected to the inflatable element can be manipulated to be in a rolled-out state such that a distal end of the inflatable element can be positioned at a distal end of the catheter. This can be achieved as described herein, e.g. by moving an inner and/or outer element of the catheter as described herein and/or by inflating the rolling membrane.

For example, the step of delivering can include delivering the medical device and/or substance along a distal direction, preferably to a distal end of the catheter.

The method can, in some examples, include the step of deflating the inflatable element. This step can preferably occur after the step of delivering (if present).

The method can further include the step of retracting the catheter from the vessel. Preferably this step can be carried out after the step of deflating the inflatable element. The step of retracting can include moving the inner shaft and/or the outer shaft of the catheter proximally and/or deflating the rolling membrane.

While specific embodiments of the present invention have been shown and described, it should be understood that other modifications, substitutions and alternatives are apparent to one of ordinary skill in the art. Such modifications, substitutions and alternatives can be made without departing from the spirit and scope of the invention, which should be determined from the appended claims.

Various features of the invention are set forth in the appended claims.

1. A catheter comprising:
an outer shaft, an inner shaft, a rolling membrane and an inflatable element comprising an inflatable volume, wherein the inner shaft comprises an inner shaft lumen and the inner shaft is at least partially arranged within the outer shaft, wherein the rolling membrane is connected to the outer shaft, and wherein the inflatable element comprises at least two proximal ends connected to the inner shaft and at least one distal end connected to the rolling membrane.

2. The catheter of claim 1, wherein the inflatable element, at least when inflated, extends along a longitudinal axis (L) of the catheter by 20 to 40 cm.

3. The catheter of claim 1, wherein the inflatable element is configured such as to provide a wall thickness, when inflated, of about 0.02 to 0.5 mm.

4. The catheter of claim 1, wherein the inflatable element and/or the rolling membrane can at least partially be rolled out in a distal direction (D) of the catheter.

5. The catheter of claim 1, wherein the inflatable element comprises an outer cylindrical element and an inner cylindrical element, wherein the inner cylindrical element is at least in part arranged within the outer cylindrical element such that the inflatable volume is defined as a hollow cylinder volume therebetween.

6. The catheter of claim 5, wherein each of the inner cylindrical element and the outer cylindrical element comprises a distal end, and wherein the distal ends are connected to each other.

7. The catheter of claim 5, wherein the inner cylindrical element and the outer cylindrical element are connected to each other proximal to the at least one distal end (D) of the inflatable element.

8. The catheter of claim 5, wherein the inner cylindrical element is connected to the outer cylindrical element at at least two fixation points.

9. The catheter of claim 5, wherein the inner cylindrical element is connected to the outer cylindrical element by at least one fixation seam arranged at least in part along a longitudinal axis (L) of the catheter.

10. (canceled)

11. The catheter of claim 1, wherein the inner shaft comprises or consists of a first tubular element and a second tubular element, wherein the first tubular element is arranged within the second tubular element.

12. The catheter of claim 21, wherein the inflatable volume of the inflatable element is in fluid communication with the annular lumen.

13. (canceled)

14. The catheter of claim 1, wherein the proximal ends of the inflatable element are connected to the distal end of the inner shaft.

15. The catheter of claim 11, wherein the inner cylindrical element is connected to the first tubular element and the outer cylindrical element is connected to a second tubular element.

16. The catheter of claim 11, wherein a proximal end of the inner cylindrical element is connected to a distal end of the first tubular element and a proximal end of the outer cylindrical element is connected to a distal end of the second tubular element.

17. The catheter of claim 1, wherein the inner shaft lumen is configured to allow a delivery of a medical drug and/or a medical device to a distal portion (D) of the catheter.

18. The catheter of claim 2, wherein the inflatable element, at least when inflated, extends along a longitudinal axis (L) of the catheter by by 26 to 35 cm.

19. The catheter of claim 3, wherein the inflatable element is configured such as to provide a wall thickness, when inflated, of about 0.1 to 0.2 mm

20. The catheter of claim 8, wherein the at least two fixation points are separated from each other by 2 to 5 mm.

21. The catheter of claim 11, wherein the first tubular element is concentrically arranged within the second tubular element thereby defining an annular lumen.
